(19) United States
(12) Patent Application Publication
(10) Pub. No.: US 2021/0015581 A1
(43) Pub. Date: Jan. 21, 2021

(54) Calibration Frame for Gamma Knife

(71) Applicant: Jonathan Tyler, Edmonton (CA)

(72) Inventor: Jonathan Tyler, Edmonton (CA)

(21) Appl. No.: 16/970,895

(22) PCT Filed: Jan. 18, 2019
(86) PCT No.: PCT/IB2019/000365

§ 371 Date: Aug. 18, 2020

Related U.S. Application Data
(60) Provisional application No. 62/619,454, filed on Jan. 19, 2018.

(51) Int. Cl. A61B 90/14; A61N 5/10

(52) U.S. Cl. CPC A61B 90/14; A61B 2090/101; A61N 5/1084

(57) ABSTRACT

The inventive alignment frame simplifies the task of attaching a fixation frame to a patient's skull prior to radiotherapy treatments. The frame is adjustably attachable to the fixation frame and is equipped with a bite plate that allows a patient to bite down on the bite plate to immobile the alignment-calibration frame and the attached fixation frame. The positional relationships are adjusted to bring the fixation screws into optimal locations on the patient's skull. Needle calibration assemblies placed into the fixation screw holders include clear needle guides through which a hypodermic needle is inserted to inject anesthetic into the future fixation screw location. The needle calibration assemblies are removed and the optimal length fixation screw is tightened into the anesthetized location. After all the fixation screws are in place, the patient releases the bite plate and the entire alignment-calibration frame is removed from the fixation frame.

### CROSS-REFERENCE TO PRIOR APPLICATIONS

The present application is the United States National Stage of PCT International Application No. PCT/IB2019/000365, with an International Filing Date of Jan. 18, 2019, which is based on and claims the benefit and priority of U.S. Provisional Patent Application No. 62/619,454 filed on 19 Jan. 2018.

### U.S. GOVERNMENT SUPPORT

Not Applicable

### BACKGROUND OF THE INVENTION

### Area of the Art

The present invention is in the area of surgery and oncology and is more specifically directed to an improved positioning device for use with high intensity radiosurgery devices.

### Description of the Background

Stereotactic Radiosurgery (SRS) involves the destruction of selected tissue in the brain or spine using ionizing radiation, rather than excision using standard surgical procedures. The technique was pioneered by the Swedish neurosurgeon Lars Leksell in 1949, and introduced clinically in 1984. The basic idea is to provide a plurality of gamma ray beams (although beams of a variety of different radiation types can be used) which can be controlled and aimed with great accuracy. The beams are arranged so that many beams intersect at the target (tumor or other abnormality) where the combined beams provide sufficient energy to ablate or otherwise destroy the target tissue. Because the surrounding tissues receive energy from only a single beam, they are relatively unharmed. One such source of multiple beams is the “gamma knife” in which a plurality of controllable radiation sources are located in a large instrument that appears not unlike a conventional magnetic resonance imaging (MRI) machine. The patient is placed on a treatment bed, which is moved into the hemispherical vault of the “gamma knife.” Thereafter, the “trick” is to maneuver/control the beams to intersect only within the target tissue that may be as small as a millimeter in diameter.

The term “stereotactic” refers to the implementation of a three-dimensional coordinate system to permit a direct correlation between the virtual images obtained through diagnostic procedures such as MRI or computed tomography (CT) and the actual target structure in the patient's brain. The development of the Leksell stereotactic frame has made targeting extremely precise, permitting the inactivation or eradication of brain lesions with minimal effect on the surrounding normal tissue.

The Leksell stereotactic system, (the “fixation frame”), consists of an aluminum ring that is securely affixed to the patient's skull with at least three fixation screws (four screws are used in the majority of cases). The fixation frame both immobilizes the skull and serves as a reference frame by which the diagnostic images and the actual tumor are superimposed. Once attached to the patient and locked into the frame support of the treatment table, the fixation frame provides unprecedented precision for radiosurgery. The patient is moved into the gamma knife vault and precisely positioned using the fixation frame to provide reference so that the gamma knife's isocenter (the point of intersecting radiation beams) is coincident with the target. Treatment consists of exposure of the target to as many as 192 hemispherically or radially arrayed, computer-controlled ⁶⁰Cobalt sources or “seeds” from which radiation is focused on the lesion without exposing surrounding brain tissue to excessive radiation. Accordingly, absolute fixation of the patient's skull in the frame, and immobilization of the frame onto the gamma knife treatment table, are critical to safe and effective treatment.

The positioning of the Leksell fixation frame in relation to the gamma knife is superb, but a major difficulty remains in properly affixing the frame to the patient's skull by (usually four) bone screws which lock the frame to the skull. To limit patient discomfort, a local anesthetic is injected into each fixation point prior to screwing in the bone screw. Today fixation is a cumbersome process, usually involving several people and a great deal of time and effort on the part of surgical support staff in a trial and error determination of injection and attachment points for the fixation frame.

Positioning the frame on the patient's head is a difficult and time-consuming procedure, requiring three or four people—one to hold the patient's head, another to hold the frame in the appropriate position, another to mark the locations for the fixation screws and inject local anesthetic into the skin, and another to select the appropriate fixation screws and screw them into the patient's skull at the selected points to lock the patient's head into the frame. Because the patient's head and the frame are essentially free-floating with respect to each other, it is difficult to ascertain the correct position for injection of the anesthetic, and frequently more than one injection per site is required. In addition, the length of each fixation screw is critically important, and must be chosen with a precision of ±2 mm to be effective—too short and the screws will not be able to clamp the skull securely, too long and the screws will interfere with structures inside the close confines of the gamma knife's treatment vault when the patient is moved into position.

With the frame held only by hand, it is extremely difficult to gauge the length of screw required in each of the three-four positions and fixation screws that have already been tightened into the skull may need to be removed and replaced with screws of different lengths during the fixation procedure. Leksell has attempted to overcome some of these problems by incorporating an optional positioning aid into the fixation frame. This consists of two sliding supports, each equipped with a probe that can be pushed into one of the patient's ear canals. However, this is not a particularly satisfactory solution, as it is uncomfortable for the patient to have a probe inserted into each ear canal, and the degree of stability afforded by these probes is not adequate to immobilize the frame on the head. Furthermore, ear probes are incapable of supporting the weight of the frame. At best, they assist in placing the frame in an approximate position during the fixation procedure.

Gamma knife staff in centers throughout the world have developed their own strategies to mitigate these issues. Some solutions involve Velcro® (brand of hook in loop fastener) straps or elastic bands placed over the patient's head to support the frame loosely, while others use surgical sponges or towels to pack between the frame and the patient's head during the fixation process. These are not optimal solutions, but merely make a difficult job somewhat less difficult. Nor do they significantly reduce the number of staff or the time required to perform the fixation procedure.

### SUMMARY OF THE INVENTION

An add-on device greatly simplifies the task of attaching a fixation frame to a patient's skull prior to radiotherapy treatments. Traditionally, a fixation frame is placed over the head of a patient and then attached thereto by fixation screws, which penetrate the patent's skin and press firmly on the bone of the patient's skull. Precise positioning of the frame prior to tightening the fixation screws is tedious at best. The inventive alignment-calibration frame is adjustably attachable to the fixation frame. The alignment-calibration frame is equipped with a bite plate that allows a patient undergoing treatment to bite down on the bite plate to immobile the alignment-calibration frame and the attached fixation frame.

At that point, it is possible to adjust the traverse relationship between the alignment frame and the fixation frame as well as the angular relationship between the bite plate and the alignment frame to bring the fixation screw holders on the fixation frame into optimal locations on the patient's skull. To facilitate this process, needle calibration assemblies are place into the fixation screw holders. Each needle calibration assembly includes a clear needle guide, which is brought into contact with the patient's skin. A hypodermic needle is then inserted through the guide to inject anesthetic into the future fixation screw location. The needle guide includes a calibration scale that reads out the optimal length for the fixation screw for each location. The needle calibration assemblies are then removed and the optimal length fixation screw is tightened into the anesthetized location. After all the fixation screws are in place, the patient is allowed to release the bite plate and the entire alignment-calibration frame is removed from the fixation frame.

### DESCRIPTION OF THE FIGURES

FIG. 1 is a view of a fixation frame;

FIG. 2 is a close-up view showing the optional ear clamp positioning aid;

FIG. 3 is close-up view showing the optional positioning aid with an ear probe inserted therein;

FIG. 4 is a close-up view showing how the optional positioning aid can be used to attach the calibration frame to the fixation frame;

FIG. 5 is a close-up view of the bite plate;

FIG. 6 is close-up view showing the details of two of the needle calibration assemblies;

FIG. 7 is drawing of the fixation frame and the calibration frame not interconnected;

FIG. 8 is drawing of the fixation frame and the calibration frame interconnected;

FIG. 9 is a view of the inventive alignment and calibration frame including the bite plate;

FIG. 10 is a view of fixation screws showing the different lengths;

FIG. 11 is a drawing of the alignment and calibration frame attached to the fixation frame by means of the optional ear probe support;

FIG. 12 is a drawing of the alignment and calibration frame attached to the fixation frame by means of a custom sliding clamp;

FIG. 13 is a view of the combination alignment-calibration and fixation frame positioned on a mock patient's head as seen from the front before the bite plate is inserted into the patient's mouth;

FIG. 14 is a view of the combination alignment-calibration and fixation frame positioned on a mock patient's head as seen from the front after the bite plate is inserted into the patient's mouth;

FIG. 15 is a view of the combination alignment-calibration and fixation frame positioned on a mock patient's head as seen as a perspective view from the upper left front showing the needle calibration assemblies in place;

FIG. 16 is a view of the combination alignment-calibration and fixation frame positioned on a mock patient's head as seen as a perspective view from the above showing both a needle calibration assembly and a fixation screw;

FIG. 17 is a view of the combination alignment-calibration and fixation frame positioned on a mock patient's head as seen from the rear;

FIG. 18 is a view of the combination alignment-calibration and fixation frame, with needle calibration assemblies, positioned on a mock patient's head as seen from the above;

FIG. 19 is a view of part of the combination alignment-calibration and fixation frame, with a needle calibration assembly, positioned on a mock patient's head and showing how the calibration frame is fastened to the optional positioning aid; and

FIG. 20 is an exploded view of the parts of the alignment and calibration frame.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an alignment and calibration frame for accurate and rapid fixation of a gamma knife fixation frame to a patient's skull.

The alignment frame addresses the shortcomings to the current procedure: the purpose of the invention is to streamline the fixation process for the Leksell frame; to minimize discomfort for the patient, and to reduce the time and staff required to attach the fixation frame. The frame consists of two components—(1) the alignment and calibration frame itself, and (2) needle guides.

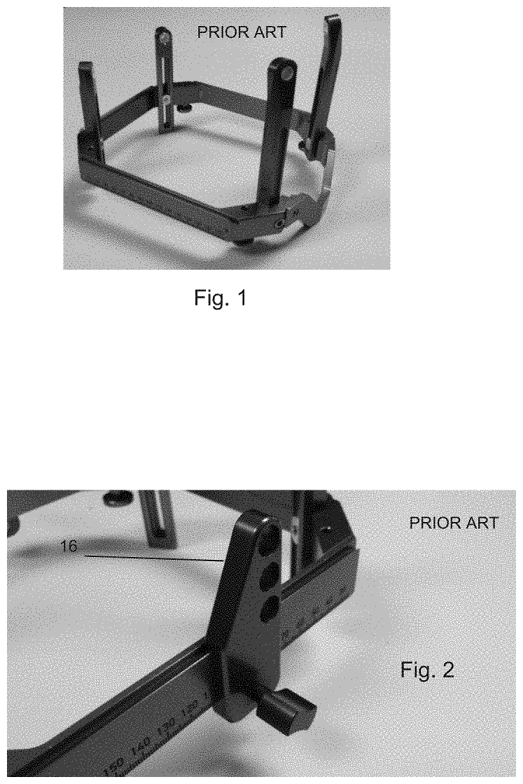

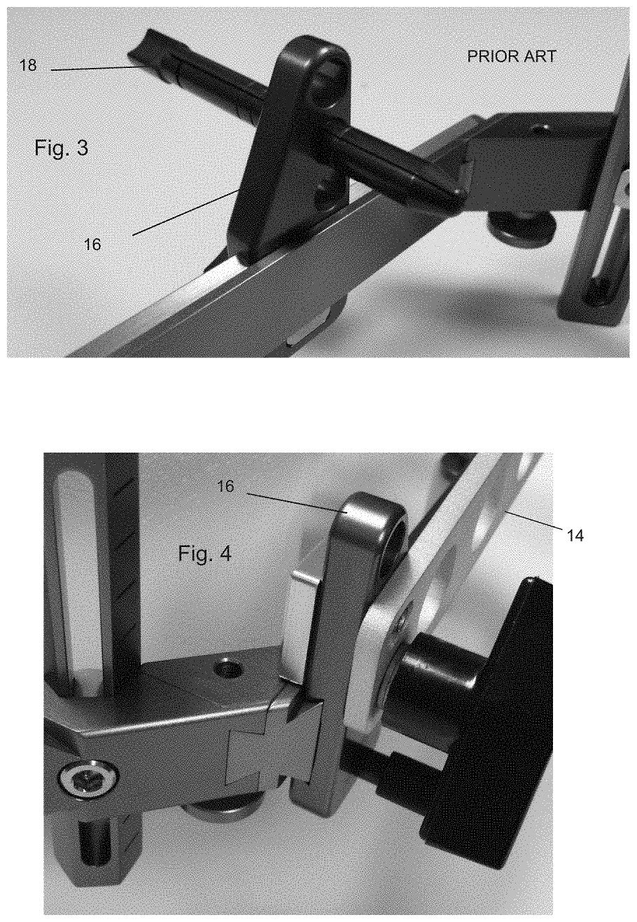

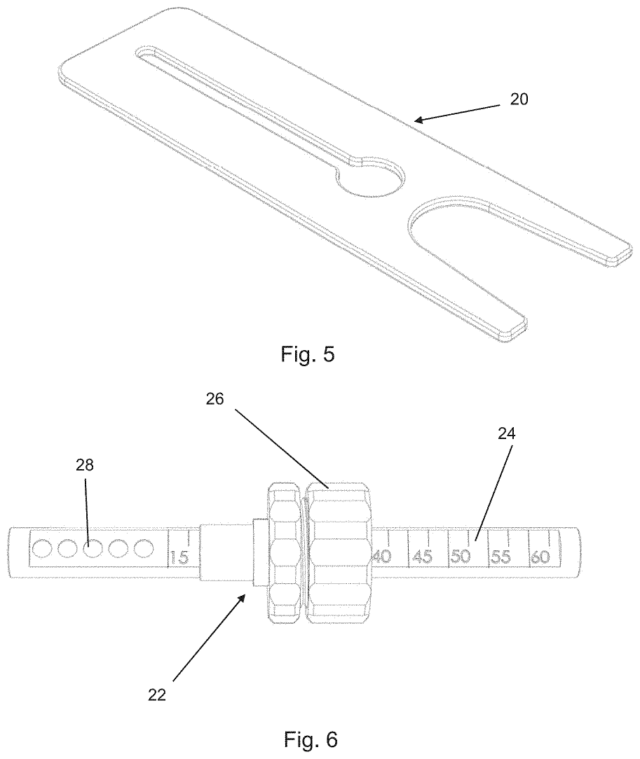

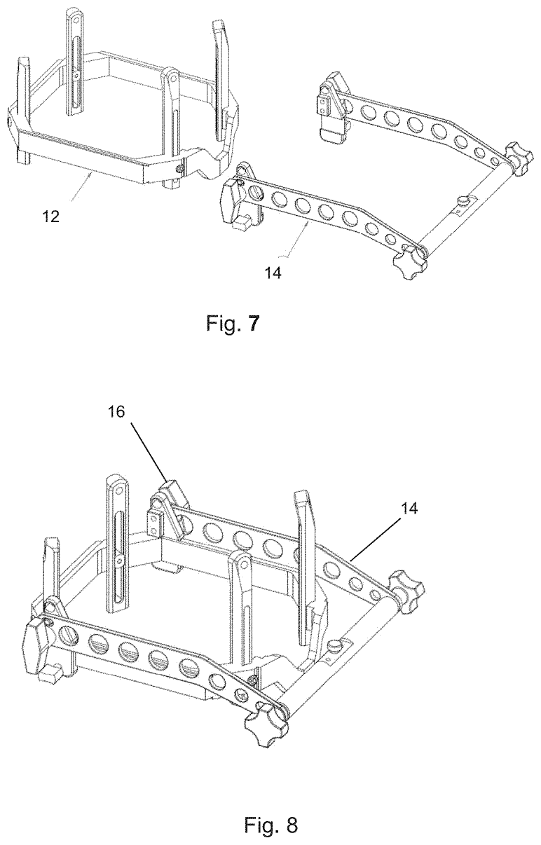

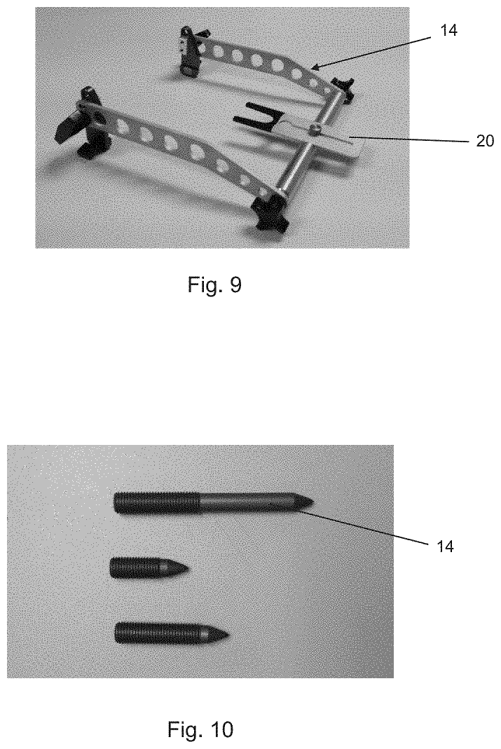

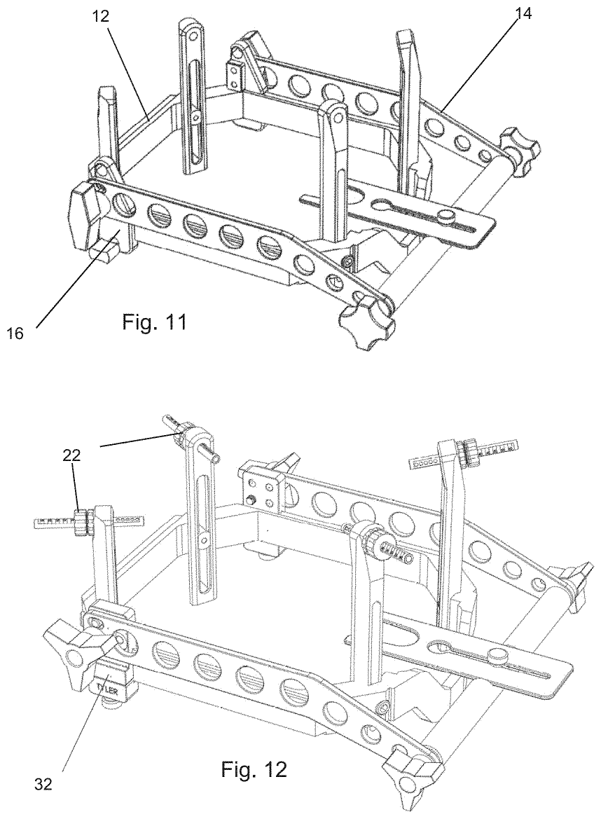

In the drawings, the shaded (gold anodized) components represent the original Leksell frame (see FIG. 1 which shows the prior art fixation frame). All other components (e.g. aluminum struts, bars, sleeves and plate; black knobs and clear tubes) are the subject of this application and are referred to collectively as the “alignment and calibration frame,” “alignment-calibration frame” or just “alignment frame.” FIG. 7 show the fixation frame 12 and the alignment-calibration frame 14 not interconnected. FIG. 8 shows the alignment frame attached to the fixation frame by way of an optional ear alignment support 16.

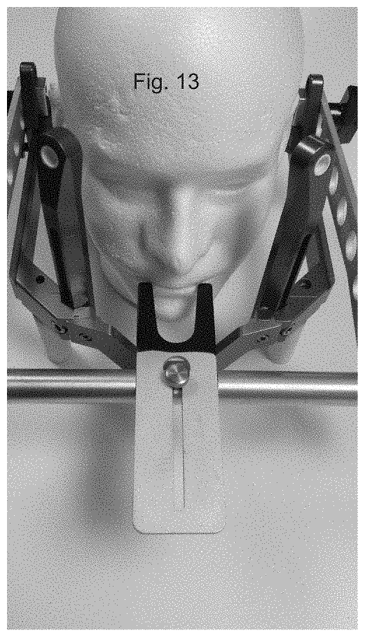

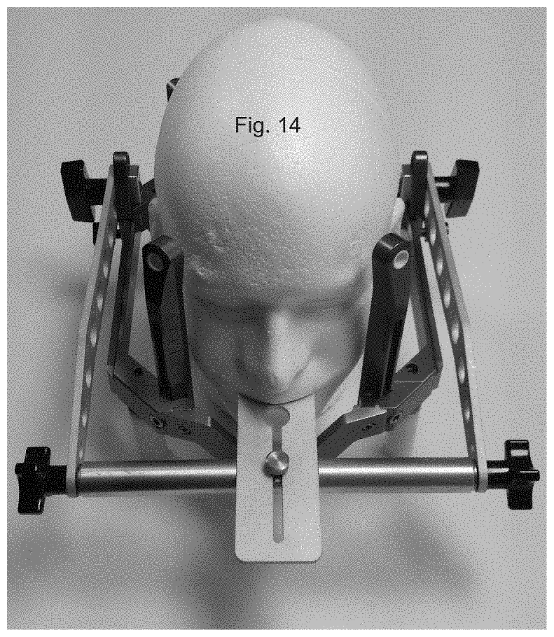

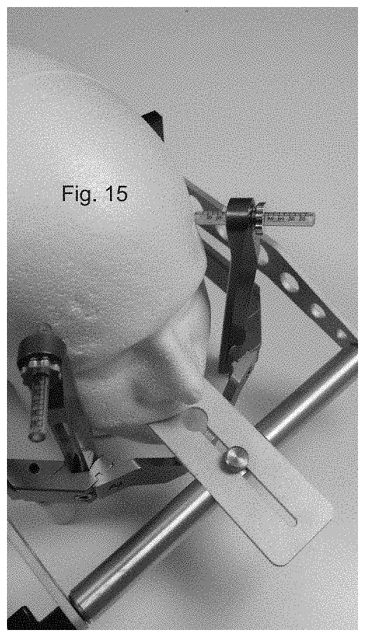

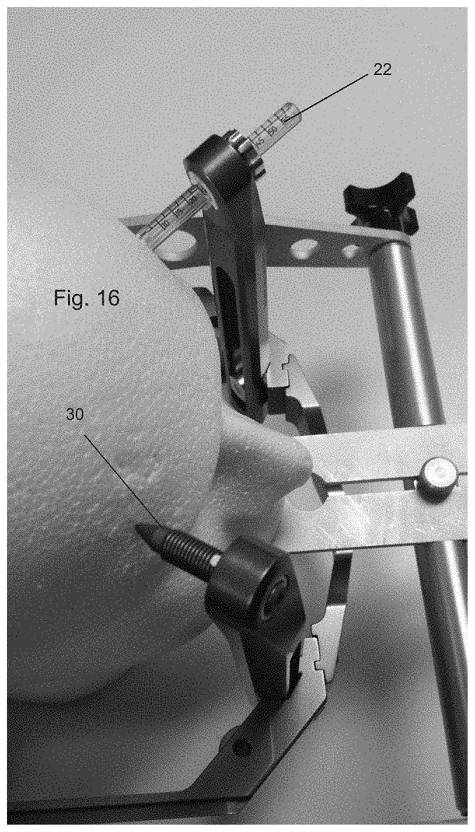

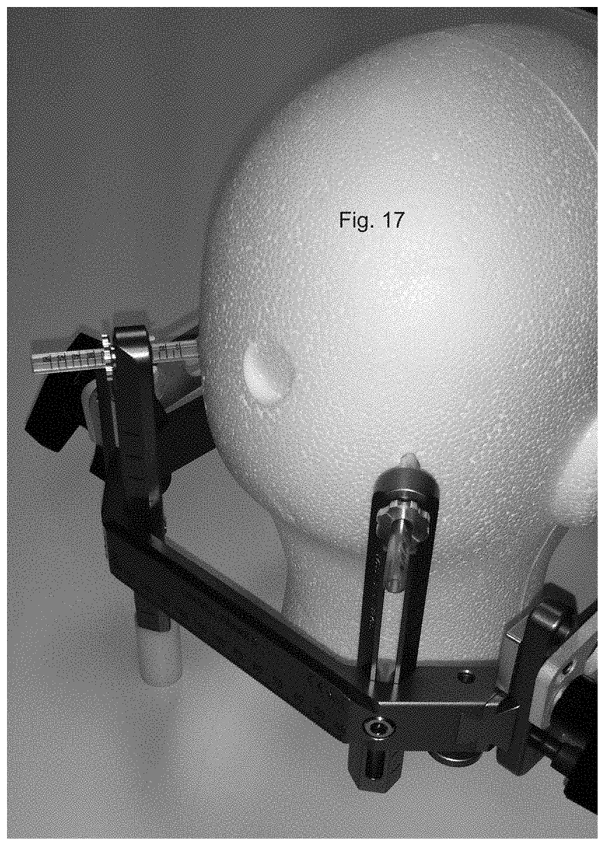

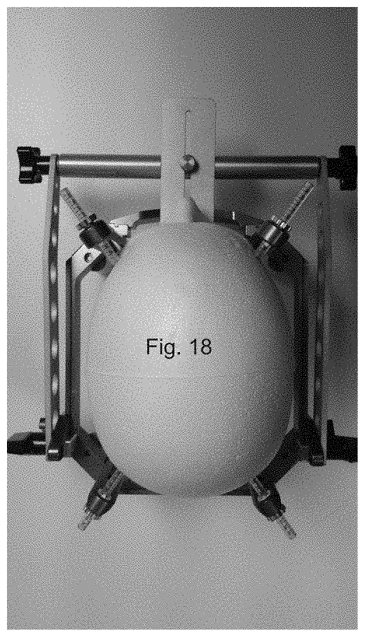

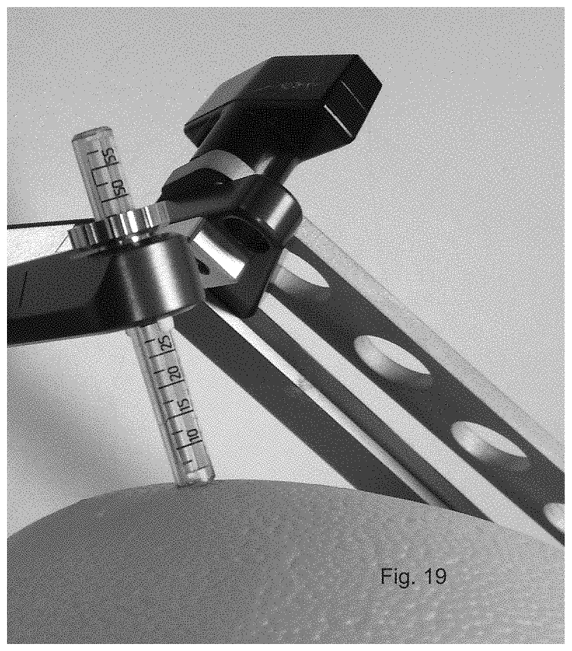

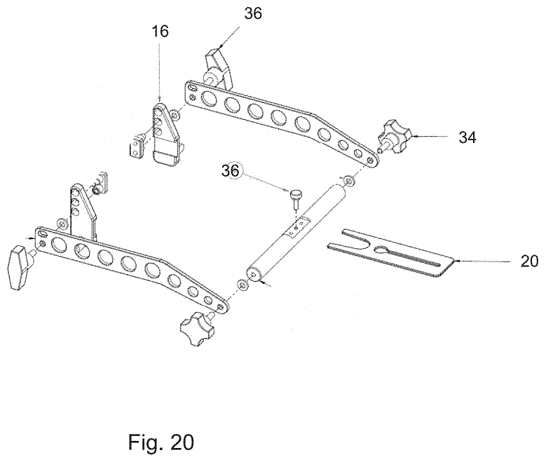

The upper jaw is part of the skull, whereas the lower jaw is attached to the skull with some of the strongest muscles in the human body. The jaw, therefore, can serve both as a reference position and as a clamp to position the fixation frame. Through a series of flexible joints in the alignment frame, a disposable bite plate can be incorporated into the fixation frame. In the illustrated embodiment, the alignment frame can be connected to the Leksell fixation frame by means of the fasteners intended for attachment of the optional sliding ear probe supports 16 or, alternatively, a dedicated dove-tail, sliding clamp 32 (see FIG. 12) or similar mechanical attachment is used instead. (The Leksell ear probe supports attach to the fixation frame by means of mating dovetail slides, and the illustrated embodiment of the alignment frame is connected to the these supports by means of contoured nuts that replace the ear probes. Alternatively, the alignment-calibration frame can be directly attached to the fixation frame by means of a sliding assembly that mates with the existing dovetails on the fixation frame).

The combined fixation frame and alignment frame is then placed over the patient's head, the bite plate 20 is moved and locked into position by means of a single thumb screw 36, and the patient is instructed to bite down firmly on the bite plate. Operating room personnel are always concerned about airway obstruction, and the potential need for an emergency intubation. The design of the bite plate 20 permits its rapid removal in the event of such an emergency—the thumbscrew 36 can readily be loosened by half a turn and the bite plate can then be retracted fully. It can then be lifted completely out of the frame if necessary because of the keyhole at the end of the slot.

After the patient bites firmly on the bite plate 20, the frame is adjusted with the flexible joints in a mechanically fluid configuration, and the joints of the alignment frame are then immobilized at the appropriate positions by turning each of four knobs (bite plate adjustment knobs 34 and fixation frame connection knobs 38). Two knobs 38 (two-arm knobs in some of the figures) adjust the angle of the connection between the fixation frame and the alignment-calibration frame in the transverse or horizontal plane, and two bite plate knobs 34 (the four-arm in some figures) adjust the position of the bite plate relative to the calibration frame, so that any required angle in the transverse plane can be accommodated. The frame is thereafter locked firmly in place and maintained in the proper position by the bite plate for subsequent fixation, regardless of whether the patient moves his head.

The needle calibration assemblies 22 can then slipped into the threaded inserts in the upright arms of the fixation frame 12. The needle calibration assemblies 22 consist of a collet portion 26 for holing the clear guide tubes 25 and for interacting with the threaded inserts on the fixation frame 12. Next, the hollow, transparent needle guides are pushed gently into firm contact with the patient's skin. Alternatively, it may be advantageous to insert the needle calibration assemblies 22 into the frame before the frame is placed over the patient's head. After the clear tubes 25 of the needle calibration assemblies 22 are brought into contact with the patient's skin, a hypodermic needle is inserted into the needle guide 25 and anesthetic is injected into the patient's skin at the precise positions at which the retaining screws 30 will subsequently be placed into the skull. The ends of the tubes 25 are equipped with needle viewing ports 28 so one can readily view the needle tip before and during penetration of the patient's skin. At the same time, the correct length of retaining screw for each position is easily determined by reading the scale 24 on the needle calibration assemblies. The needle calibration guides are then removed and the appropriate retaining screws inserted into the threaded inserts and tightened securely. Finally, the alignment frame is removed. If the frame is attached through the ear clamps, this is achieved by loosening the two cam locks, which attach it to the fixation frame by means of the ear, support slides whereupon the alignment frame can be slid sliding forward away from the patient's head and the fixation frame. Alternatively, as shown in FIG. 12, a special sliding clamp has replaced the ear clamps and is used to attach the alignment frame to the fixation frame. The entire procedure of positioning and affixing the frame to the patient can easily be handled by a single person, and in a fraction of the time required for fixation of the Leksell frame alone.

The following claims are thus to be understood to include what is specifically illustrated and described above, what is conceptually equivalent, and what can be obviously substituted. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

What is claimed is:

1. An alignment and calibration frame for use in attaching a fixation frame to a patient's skull comprising:
attachment means for removably attaching the alignment and calibration frame to the fixation frame;
a bite plate adjustably connected to the alignment and calibration frame whereby the patient can immobilize the alignment and calibration frame by biting down on the bit plate;
needle calibration assemblies for pin pointing sites for fixation screw insertion; and
adjustment means for adjusting a position of the calibration frame relative to the fixation frame.

2. The alignment and calibration frame of claim 1, wherein the attachment means attach to an adjustable ear clamp which is an optional part of the fixation frame.

3. The alignment and calibration frame of claim 1, wherein the attachment means comprise a sliding clamp that attaches to the fixation frame in place of an adjustable ear clamp.

4. The alignment and calibration frame of claim 1, wherein the needle calibration assemblies include markings that correspond to a correct length of a fixation screw.

5. The alignment and calibration frame of claim 1, wherein the adjustment means comprise adjustment knobs for controlling connection between the fixation frame and the alignment and calibration frame in a horizontal or transverse plane when the two frames are connected.

6. The alignment and calibration frame of claim 1, wherein the adjustment means comprise adjustment knobs for controlling position of the bite plate relative to the alignment-calibration frame.
